(19) United States
(12) Patent Application Publication
(10) Pub. No.: US 2026/0042795 A1
(43) Pub. Date: Feb. 12, 2026

(54) DIPROPYLAMINE AS BASE FOR THE USE IN FMOC DEPROTECTION IN SOLID-PHASE PEPTIDE SYNTHESIS

(71) Applicant: UNIVERSITÄT BERN, Bern (CH)

(72) Inventors: Jean-Louis REYMOND, Bern (CH); Hippolyte PERSONNE, Bern (CH); Thissa N. SIRIWARDENA, Bern (CH); Sacha JAVOR, Bern (CH)

(73) Assignee: UNIVERSITÄT BERN, Bern (CH)

(21) Appl. No.: 19/100,568

(22) PCT Filed: Aug. 1, 2023
(86) PCT No.: PCT/EP2023/071334

§ 371 Date: Feb. 2, 2025

(30) Foreign Application Priority Data
Aug. 1, 2022 (EP) 22188039.6

(51) Int. Cl. C07K 1/06; C07K 1/04; C07K 1/08

(52) U.S. Cl. CPC C07K 1/063; C07K 1/042; C07K 1/08

(57) ABSTRACT

The invention relates to a method for the preparation of peptides via solid-phase peptide synthesis and particularly to a method of deprotecting of an Fmoc protected amino acid building block linked to a resin R-AA-(AA)ₙ-PF.

The present invention relates to an optimised method for fluorenylmethoxycarbonyl (Fmoc) deprotection of peptides in solid-phase peptide synthesis (SPPS).

### BACKGROUND OF THE INVENTION

Solid-phase peptide synthesis (SPPS) is a widely used method for the synthesis of peptides, wherein amino acids are covalently bound to a solid support material and peptide growth occurs step-by-step utilising a selective protecting group strategy. In comparison to normal liquid state synthesis, SPPS is characterised through high efficiency and high throughput as well as increased simplicity, speed and yields. Amino acids are protected at all reactive functional groups present, forming amino acid building blocks, whereby the order of reaction of each functional group can be controlled through selective deprotection. In a basic method of SPPS, the first amino acid building block is linked to a resin at either its C-terminus or N-terminus, usually its C-terminus. The two most used strategies are fluorenylmethoxycarbonyl (Fmoc)-SPPS and tert-butyloxycarbonyl (Boc)-SPPS, wherein the N-terminus is protected with either Fmoc or Boc. In the next step the building block N-terminus is deprotected in a deprotection step, yielding a free amine. The formation of a peptide bond requires a carboxylic acid as reaction partner for the free amine. The building block to be attached to the free amine is thus protected at its N-terminus and the C-terminus has to undergo an activation step. The resin linked free amine and the activated carboxylic acid then form a peptide bond in a coupling step, yielding an N-terminus protected dipeptide in the case of single amino acid building blocks. The N-terminus protected dipeptide is then deprotected again to be coupled to another C-terminus activated amino acid building block. This cycle is repeated to form the desired peptide. Once the desired peptide length is reached, all side chains are deprotected and the peptide is cleaved from the resin (FIG. 1).^{R1}

SPPS with Fmoc as α-amino protecting group for amino acid building blocks is currently the dominant synthesis method for peptide research and manufacturing. Piperidine (PPR) serves as the optimal Fmoc removal reagent in SPPS because it acts both as an efficient base to trigger β-elimination of carbamic acid and as nucleophile to quench the reactive dibenzofulvene by-product (FIG. 2a). However, PPR is expensive, stinky, and highly regulated due to its use in illegal drug manufacturing. PPR can be replaced by a non-stink but still expensive mixture of piperazine (PZ) as nucleophilic quencher and 1,8-diazabicyclo[5.4.0]undec-7-ene (DBU) as base (FIG. 2b). However, both PPR and PZ/DBU trigger the formation of aspartimide in some aspartic acid containing sequences, which can hydrolyze to α- or β-peptides (FIG. 2c). Adding weak acids such as formic acid or ethyl cyanohydroxyiminoacetate (Oxyma) to temper the basicity of the PPR solution reduces aspartimide formation, however this does not solve the cost, stink and availability issues of PPR. Several reagents or aspartate side chain protecting groups have been reported in attempts to overcome the limitations of PPR or PZ/DBU, however none of them combines low cost and convenient use with high yields and low aspartimide.^{R2-R8}

Based on the above-mentioned state of the art, the objective of the present invention is to provide means and methods to use optimised reaction conditions in the deprotection of Fmoc protected peptides with low costs and great availability. This objective is attained by the subject-matter of the independent claims of the present specification, with further advantageous embodiments described in the dependent claims, examples, figures and general description of this specification.

### SUMMARY OF THE INVENTION

A first aspect of the invention relates to a method for the preparation of peptides via solid phase peptide synthesis, wherein
an Fmoc protected amino acid building block linked to a resin R-AA-(AA)ₙ-PF, with
   R being a resin
   AA being an amino acid building block
   PF being an Fmoc protecting group
   n being the number of coupling cycles,
is deprotected in a deprotection step prior to a coupling step, using dibutylamine or dipropylamine, yielding R-AA-(AA)ₙ, wherein
in said coupling step, another amino acid building block AA-P is coupled to R-(AA)-(AA)ₙ, yielding R-AA-(AA)ₙ₊₁-P until a final coupling cycle n_{end}, wherein P is a protecting group of the amino acid building block AA at the N-terminus or PF.

### Terms and Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising,” “having,” “containing,” and “including,” and other similar forms, and grammatical equivalents thereof, as used herein, are intended to be equivalent in meaning and to be open-ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictates otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic, and biochemical methods (see generally, Sambrook et al., Molecular Cloning: A Laboratory Manual, 4th ed. (2012) Cold Spring Harbor Laboratory Press, Cold Spring Harbor, N.Y. and Ausubel et al., Short Protocols in Molecular Biology (2002) 5th Ed, John Wiley & Sons, Inc.) and chemical methods.

The term resin R in the context of the present specification relates to a solid support. Resins are typically small and spherical beads, of two different sizes: 100-200 mesh (75-150 microns) and 200-400 mesh (35-75 microns), comprising a polymer. The most common resin in solid-phase chemistry is polystyrene (PS) which is often supplemented with divinylbenzene (PS-DVB). Other resins commonly used are polyamines and polyethylenglycol-polystyrene (PEG-PS) resins.

Resins are further functionalised with a linker. For Fmoc-SPPS, common linkers are Rink amide, Wang, hexamethylphosporamide (HMPA), hexamethylenebisacetamide (HMBA), 4-(4-hydroxymethyl-3-methoxyphenoxy)-butyric acid (HMPB), 2-Chlorotrityl, super acid sensitive resin (SASRIN), Rink acid, Hydrazine, or sulfonamide. For Boc-SPPS commonly used linkers are Merrifield, PAM or MBHA.^{R9}

The term amino acid building block AA in the context of the present specification relates to a single amino acid or short peptide comprising two to three amino acids, which are protected at their amino acid side chain(s) with any common protecting group used for amino acids.^{R10}

The term n in the context of the present specification relates to the number of coupling cycles in solid-phase peptide synthesis. The minimum number of coupling cycles is 1. The maximum number of coupling cycles is not defined.

The term coupling cycle in the context of the present specification relates to the step in solid-phase peptide synthesis in which the deprotected, resin linked amino acid building block R-AA-(AA)ₙ is coupled with an activated amino acid building block AA-P.

The term n_{end} in the context of the present specification relates to the final coupling cycle in solid-phase synthesis, wherein the final coupling cycle is reached once the desired peptide length is reached. Once the final desired peptide length is reached, the peptide is fully deprotected at its N-terminus and side chains, cleaved from the resin and purified if desired.

The term P in the context of the present specification relates to a protecting group at the amino acid building block N-terminus, wherein the protecting group may be any common protecting group used in peptide synthesis generally known by a person skilled in the art, including benzyl amine (NBn), N-carboxybenzyl (Cbz), tert-butyloxycarbonyl (Boc), allyloxycarbonyl (Alloc), methyltrityl (Mtt), 1-(4,4-dimethyl-2,6-dioxocyclohex-1-ylidene)ethyl (Dde), 1-(4,4-dimethyl-2,6-dioxocyclohexylidene)-3-methylbutyl (ivDde) or fluorenylmethoxycarbonyl (Fmoc) (see Clayden, Greeves, Warren and Wothers, Organic Chemistry, 2001, p. 657).

The term PF in the context of the present specification relates to the fluorenylmethoxycarbonyl (Fmoc) protecting group. Fmoc is a base-labile and acid-stable protecting commonly used in peptide synthesis. Fmoc is usually introduced through 9-fluorenylmethyloxycarbonyl chloride (Fmoc-CI) (see Clayden, Greeves, Warren and Wothers, Organic Chemistry, 2001, p. 656-658).

The term polypeptide in the context of the present specification relates to a molecule consisting of 50 or more amino acids that form a linear chain wherein the amino acids are connected by peptide bonds. The amino acid sequence of a polypeptide may represent the amino acid sequence of a whole (as found physiologically) protein or fragments thereof. The term “polypeptides” and “protein” are used interchangeably herein and include proteins and fragments thereof. Polypeptides are disclosed herein as amino acid residue sequences.

The term peptide in the context of the present specification relates to a molecule consisting of up to 50 amino acids, in particular up to 30 amino acids, more particularly up to 15 amino acids, that form a linear chain wherein the amino acids are connected by peptide bonds.

Amino acid residue sequences are given from amino to carboxyl terminus. Capital letters for sequence positions refer to L-amino acids in the one-letter code (Stryer, Biochemistry, 3^{rd} ed. p. 21). Lower case letters for amino acid sequence positions refer to the corresponding D- or (2R)-amino acids. Sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (lie, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

### DETAILED DESCRIPTION OF THE INVENTION

A first aspect of the invention relates to a method for the preparation of peptides via solid phase peptide synthesis, wherein
an Fmoc protected amino acid building block linked to a resin R-AA-(AA)ₙ-PF, with
   R being a resin
   AA being an amino acid building block
   PF being an Fmoc protecting group
   n being the number of coupling cycles,
is deprotected in a deprotection step prior to a coupling step, using dibutylamine or dipropylamine, yielding R-AA-(AA)ₙ, wherein
in said coupling step, another amino acid building block AA-P is coupled to R-(AA)-(AA)ₙ, yielding R-AA-(AA)ₙ₊₁-P until a final coupling cycle n_{end}, wherein P is a protecting group of the amino acid building block AA at the N-terminus or PF.

The term amino acid building block AA relates to a single amino acid or short peptide comprising two to three amino acids, which are protected at their amino acid side chains with any common protecting group used in peptide synthesis.

In SPPS, the first amino acid building block AA-(AA)ₙ-PF is linked to a resin R, which represents the solid-phase, yielding R-AA-(AA)ₙPF. In the first amino acid building block n equals 0. The first amino acid building block R-AA-PF is then deprotected using dibutylamine or dipropylamine resulting in the deprotected amino acid building block R-AA.

In certain embodiments, at least one AA comprises aspartic acid.

Aspartic acid is the responsible amino acid in the formation of aspartimide and subsequent hydrolysis to α- or β-peptides during SPPS (FIG. 2c). The base present in the Fmoc deprotection step of a peptide deprotonates a neighbouring secondary amine to aspartic acid, which in turn reacts in a ring closure to form aspartimide. Through water, which is present in the synthesis, hydrolysis of aspartimide takes place forming either an α- or β-peptide of the produced peptide.

The method described in the present invention, wherein the use of dibutylamine or dipropylamine drastically reduces the formation of aspartimide resulting in better peptide yields and thus differs from existing procedures for SPPS.

In certain embodiments, the deprotection step is performed with dipropylamine.

The use of dipropylamine as a base for the deprotection of Fmoc protected amino acid building blocks or Fmoc protected peptides solves several problems present within the use of the commonly used bases for the Fmoc deprotection of Fmoc protected amino acid building blocks or Fmoc protected peptides, piperidine and piperazine. Both dibutylamine and dipropylamine are unregulated, inexpensive, do not stink, are readily available. Fmoc deprotection with either dibutylamine or dipropylamine lead to good peptide yields and reduce the formation of aspartimide. Use of dipropylamine still results in slightly better yields than use of dibutylamine as shown in Examples 1 to 3 of the present invention.

In certain embodiments, the used amount of dipropylamine is 10 to 50% (v/v).

In certain embodiments, the used amount of dipropylamine is 20 to 40% (v/v).

In certain embodiments, the used amount of dipropylamine is 25 to 35% (v/v).

Dipropylamine was found to work best when used as 25% (v/v) in N,N-dimethylformamide (DMF). The addition of Oxyma, an additive for carbodiimides in SPPS and known to reduce aspartimide formation did not further reduce aspartimide formation when dipropylamine was used at said volume percentage which further simplifies the procedure and saves costs.

In certain embodiments, the method is conducted at 10 to 90° C.

In certain embodiments, the method is conducted at 65 to 90° C.

The use of dibutylamine or dipropylamine in SPPS enables to conduct SPPS at higher temperatures of 65 to 90° C. The higher temperatures enable the use of dibutylamine or dipropylamine, which have high boiling points. High temperatures reduce the aggregation of peptides and swelling of the resin. Furthermore, the reaction time is reduced through an increase of the reaction rates, resulting in higher peptide yields.

In certain embodiments, the method is conducted at 75 to 90° C.

In certain embodiments, AA-P is activated in an activation step prior to said coupling step.

Another amino acid building block AA-P is coupled to R-AA-(AA)ₙ in a coupling step. For the coupling step to take place, AA-P, wherein P is a protecting group of the amino acid building block AA at its N-terminus or PF, the amino acid building block AA needs to be activated in the activation step. The activation takes places at the C-terminus of the amino acid building block using common coupling reagents. Common coupling reagents are N,N′-diisopropylcarbodiimide (DIC), dicyclohexylcarbodiimide (DCC), 1-hydroxybenzotriazole (HOBt), 2-(1H-benzotriazol-1-yl)-1,1,3,3-tetramethyluronium-hexafluorophosphate (HBTU), O-(7-azabenzotriazol-1-yl)-N,N,N′,N′-tetramethyluronium-hexafluorophosphate (HATU), tetra-methylfluoroformamidinium hexafluorophosphate (TFFH) or benzotriazol-1-yl-oxytri-pyrrolidinophosphonium-hexafluorophosphate (PyBOP).

In certain embodiments, the deprotection step and the coupling step can be repeated until a desired peptide length is reached, which is not more than 60 amino acids.

In certain embodiments, n_{end} is the final coupling cycle at which the desired peptide length is reached.

n_{end} does not equal the number of amino acids in the peptide as amino acid building blocks can contain 1 to 3 amino acids.

In certain embodiments, the method comprises a final deprotection step after n_{end} coupling cycles, wherein R-AA-(AA)_{nend}- P is deprotected in said deprotection step yielding R-AA-(AA)_{nend}.

In the final deprotection step, all present protecting groups at the N-terminus and side chains are removed, yielding a fully deprotected peptide which is still coupled to the resin.

In certain embodiments, the method comprises a cleavage step after n_{end} coupling cycles, wherein R-AA-(AA)_{nend} is cleaved from the resin R, yielding AA-(AA)_{nend}.

In certain embodiments, the method comprises a purification step, wherein AA-(AA)_{nend} is purified.

The peptide is separated from impurities during the purification step. Common methods for peptide purification are RP-HPLC, flash chromatography, ion-exchange chromatography, hydrophobic interaction chromatography, gel filtration chromatography, size exclusion chromatography, hydrophilic interaction chromatography and solid-phase extraction.^{R11,R12}

In certain embodiments, P is PF.

The formation of aspartimide through aspartic acid may occur on all subsequent deprotections throughout the peptide synthesis. It is thus an advantage to use solely Fmoc as N-terminal protecting group throughout the whole peptide synthesis. Furthermore, using Fmoc in all building blocks as N-terminal protecting group facilitates the synthesis especially with regard to side chain protecting group chemistry. A consistent N-terminal protecting group at all AA-P throughout the peptide synthesis allows the choice of amino acid side chain protecting groups which are stable throughout the whole synthesis. For example, Fmoc is base labile, and so purely base stable protecting groups can be used at amino acid side chains.

The invention is further illustrated by the following examples and figures, from which further embodiments and advantages can be drawn. These examples are meant to illustrate the invention but not to limit its scope.

### DESCRIPTION OF THE FIGURES

FIG. 1 Solid-phase peptide synthesis (SPPS) cycle with a) deprotection step, b) activation step, c) coupling step, d) final deprotection step, e) cleavage step; AA-P may be different amino acid building blocks in each cycle;.

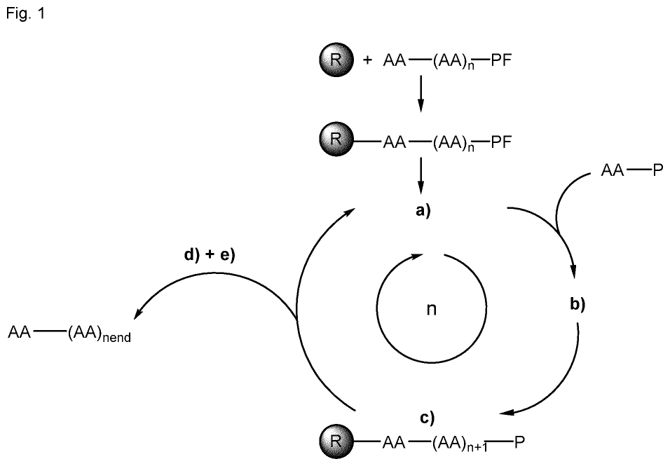

FIG. 2 shows (a) Mechanism of Fmoc (1) deprotection and trapping of dibenzofulvene (2). (b) Structural formulae of reagents used in Fmoc removal; PPR (3), PZ (4), DBU (5), Oxyma (6), DBA (7). (c) Mechanism of aspartimide (9) formation and its hydrolysis to α-peptide (10) or β-peptide (11) during SPPS; 8=tBuOH, B=base, Nu=nucleophile.

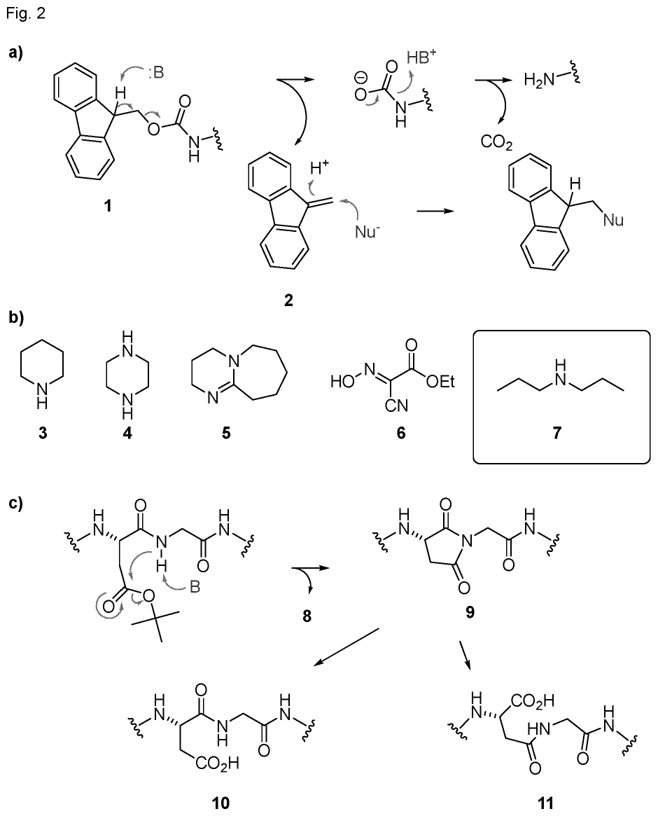

### EXAMPLES

### Example 1: Synthesis of the First and Second Generation Peptide Dendrimers, G1KL and G2KL, Via SPPS

In an initial aim to develop a high temperature (60° C.) protocol for solid-phase peptide synthesis of G1KL, using Oxyma and N,N′-Diisopropylcarbodiimide (DIC), wherein DIC acts as coupling reagent and Oxyma as acidic additive to suppress the base-induced side reaction, and DMF as solvent, the inventors identified dipropylamine (DPA) as alternative base for piperidine. DPA (pKa=10.9) is only slightly less basic than piperidine (pKa=11.1) and slightly less volatile (bp (DPA)=110° C.; bp (piperidine)=106° C.). The synthesis of the first-generation peptide dendrimer whose synthesis requires both α-amino and side-chain amino Fmoc removal, resulted in similar yields (65%) using 25% DPA, to using the state-of-the-art reagents piperidine (73%) and much better yields in comparison to using piperazine and DBU (26%) (Table 1). The difference in yield between 20% and 25% DPA was significant with 30%.

The synthesis of the second generation analogue G2KL resulted in equally good yields of 46% using 25% DPA at room temperature. The increase of DPA from 20% to 25% did not result in drastically improved yields in this case (Table 1).

While DPA leads to similar yields as the state-of-the-art reagents, diisopropylamide (DIPA) did not lead to any formation of the desired product, making it an unsuitable base for deprotection.

### Example 2: Synthesis of the Hexapeptides 1 to 7

The inventors next investigated the SPPS of the aspartimide prone hexapeptides 1 to 7 (Table 1). Using piperidine for the Fmoc deprotection of the peptide afforded 17% aspartimide (Hexapeptide 1), while 20% DPA only gave 5% which is a slightly lower aspartimide formation than for adding Oxyma to piperidine, which resulted in 6% aspartimide formation (Table 1, Hexapeptide 1). The crude product NMR was compared to the independently synthesised β-peptide of VKDGYI (Hexapeptide 1; SEQ ID NO: 3), VKD(β)GYI, which would be formed upon hydrolysis of the VKDGYI aspartimide. However, no β-peptide was detected and 49% crude peptide yield (Hexapeptide 1) using 20% DPA at 60° C. was obtained. In comparison, the yield using the state-of-the-art conditions, including 20% PPR at 60° C. was 47%.

For the hexapeptide 1, the deprotection by using solely 2% DBU was accompanied with a 25% aspartimide formation.

The inventors furthermore investigated Fmoc deprotection in the SPPS of VKDGYI (SEQ ID NO: 3) with dibutylamine and diisobutylamine. However, no deprotection using diisobutylamine took place. Dibutylamine gave a similar crude peptide yield than dipropylamine at 60° C. (52% (DBA) vs. 53% (DPA)).

Yields of the hexapeptide VKEGYI (Hexapeptide 7; SEQ ID NO 9) were similar to VKDGYI (Hexapeptide 1; SEQ ID NO: 3), with 44% crude yield using 20% DPA at 60° C. and no aspartimide formation was observed. (Table 1).

Hexapeptides 2 to 6 were deprotected using 25% DPA at 60° C. and 20% piperidine at 60° C. as a comparison. Hexapeptides 2, 3 and 4 had increased yields using DPA instead of piperidine and aspartimide formation was reduced by 25 to 100%. The yields of hexapeptides 4 and 5 were slightly lower using DPA instead if piperidine and aspartimide formation remained fairly similar (Table 1).

In comparison the hexapeptide 1 was obtained at 78% crude purity with 11% aspartamide formation at 90° C. compared to 96% crude purity and 4% aspartimide formation at 60° C., showing that the deprotection at 60° C. as well as 90° C. results in highly pure peptide.

### Example 3: Synthesis of the Peptide Drug Bivalirudin

The peptide bivalirudin consist of 20 amino acids. The synthesis of this peptide using 25% DPA in DMF at 60° C. resulted in 39% isolated yield and 46% when using piperidine (Table 1). However, the results show that even with more complex peptides, DPA represents a valuable alternative to piperidine.

TABLE 1
Extended SPPS yields of peptide dendrimers and linear peptides using various
Fmoc deprotection conditions.

| Cpd. | | Crude | Crude | Isolated |
|---|---|---|---|---|
| Sequence^{a)} | Deprotection condition^{b)} | purity^{c)} % | yield^{d)} % | yield^{e)} % |
| G1KL | 20% PPR, 60° C. | 90 | 72.5 | n.d. |
| (KL)₂KKL | 5% PZ + 2% DBU, 60° C. | 97 | 26.2 | n.d. |
| (SEQ ID NO: 1) | 20% DIPA, 60° C. | 0 | 0 | n.d. |
| | 20% DPA, 60° C. | 78 | 35.2 | n.d. |
| | 25% DPA, 60° C. | 88 | 64.5 | n.d. |
| G2KL | 20% PPR, r.t. | 79 | 64.9 | n.d. |
| (KL)₄(KKL)₂KKL | 5% PZ + 2% DBU, r.t. | 74 | 53.9 | n.d. |
| (SEQ ID NO: 2) | 20% DPA, r.t. | 82 | 42.2 | n.d. |
| | 25% DPA, r.t. | 80 | 46.4 | n.d. |
| Hexapeptide 1 | 20% PPR, 60° C. | 83 (17/0) | 46.6 | 4.5 |
| VKDGYI | 20% PPR + 0.5 M Oxyma, 60° C. | 93 (6/1) | 16.8 | n.d. |
| (SEQ ID NO: 3) | 5% PZ + 2% DBU, 60° C. | 0 (0/100) | 0 | n.d. |
| | 2% DBU, 60° C. | 52 (25/23) | 25.7 | n.d. |
| | 20% DPA, 60° C. | 95 (5/0) | 49.3 | n.d. |
| | 25% DPA, 60° C. | 96 (4/0) | 52.9 | 16.0 |
| | 25% DBA, 60° C. | 93 (4/3) | 52.1 | n.d. |
| | 25% DIBA, 60° C. | 0 (0/100) | 0 | n.d. |
| | 25% DPA, 90° C. | 78 (11/11) | 33.5 | n.d. |
| Hexapeptide 2 | 20% PPR, 60° C. | 67 (32/1) | 40.9 | n.d. |
| GDGAKF | 25% DPA, 60° C. | 84 (8/8) | 49.2 | n.d. |
| (SEQ ID NO: 4) | | | | |
| Hexapeptide 3 | 20% PPR, 60° C. | 84 (8/8) | 40.3 | n.d. |
| VKDRYI | 25% DPA, 60° C. | 90 (4/6) | 43.4 | n.d. |
| (SEQ ID NO: 5) | | | | |
| Hexapeptide 4 | 20% PPR, 60° C. | 96 (3/1) | 50.6 | n.d. |
| GDRAKF | 25% DPA, 60° C. | 99 (0/1) | 62.5 | n.d. |
| (SEQ ID NO: 6) | | | | |
| Hexapeptide 5 | 20% PPR, 60° C. | 90 (5/5) | 53.1 | n.d. |
| VKDCYI | 25% DPA, 60° C. | 88 (4/8) | 48.0 | n.d. |
| (SEQ ID NO: 7) | | | | |
| Hexapeptide 6 | 20% PPR, 60° C. | 97 (1/2) | 54.7 | n.d. |
| VKDAYI | 25% DPA, 60° C. | 96 (1/3) | 51.3 | n.d. |
| (SEQ ID NO: 8) | | | | |
| Hexapeptide 7 | 20% PPR, 60° C. | 98 (0/2) | 47.7 | n.d. |
| VKEGYI | 5% PZ + 2% DBU, 60° C. | 99 (0/1) | 52.4 | n.d |
| (SEQ ID NO: 9) | 20% DPA, 60° C. | 98 (0/2) | 44.3 | n.d |
| Bivalirudin | 20% PPR, 60° C. | 77 | n.d | 46.3 |
| fPRPGGGGNG | 25% DPA, 60° C. | 77 | n.d | 38.7 |
| DFEEIPEEYL^{a)} | | | | |
| (SEQ ID NO: 10) | | | | |
| VKD(β)GYI | | | | |
| (SEQ ID NO: 11) | | | | |

^{a)}One letter code for amino-acids, D-amino acids in lower case, italic K indicates branching L-lysine, C-termini are carboxamide except for Bivalirudin which is carboxyl.
^{b)}SPPS was carried out in DMF using Oxyma/DIC as coupling reagents and the indicated base for Fmoc removal. PPR = Piperidine, PZ = Piperazine, DBU = 1,8-diazabicyclo[5.4.0]undec-7-ene, DPA = Dipropylamine, DIPA = Diisopropylamine, DBA = Dibutylamine, DIBA = Diisobutylamine. Percentages (%) are in w/v in case of PZ and in v/v otherwise.
^{c)}Crude purity for hexapeptides 1-7 is given as follow: % desired product (% aspartimide or glutarimide / % other byproducts). The crude product after resin cleavage was precipitated, washed and dried, and analyzed by analytical HPLC to determine the percentage of desired product, aspartimide and other byproducts.
^{d)}Crude yield is calculated relative to the amount of resin, its indicated loading and crude purity.
^{e)}Isolated yields were calculated after preparative RP-HPLC purification according to the amount of resin and its indicated loading.
n.d. = not determined.

### Materials and Methods

DMF (N,N-dimethylformamide) was purchased from Thommen-Furler AG, Oxyma Pure (hydroxyiminocyanoacetic acid ethyl ester) was purchased from SENN AG, 010 (N,N′-diisopropyl carbodiimide) was purchased from Iris BIOTECH GMBH, piperidine was purchased from Acros Organics, piperazine, butanol and DBU (1,8-Diazabicyclo[5.4.0]undec-7-ene) were purchased from Alfa Aesar, dipropylamine, diisopropylamine, diethylamine, dibutylamine, diisobutylamine, DMAP (4-dimethlyaminopyridine), HOBt (Hydroxybenzotriazole), DIPEA (N,N-diisopropylethylamine) and DODT (2,2′-(Ethylenedioxy)diethanethiol) were purchased from Sigma Aldrich, triisopropylsilane and TFA (trifluoroacetic acid) were purchased from Fluorochem Ltd, formic acid was purchased from Fluka Analytical. For amino acid, Fmoc-Nle-OH was purchased from Iris BIOTECH GMBH, Fmoc-Asp-OtBu and Fmoc-Glu-OtBu were purchased from Novabiochem, all the other amino acids were purchased from Shanghai Space Peptides Pharmaceuticals Co., Ltd. Chemicals were used as supplied and solvents were of technical grade. Amino acids were used as the following derivatives: Fmoc-Leu-OH, Fmoc-Lys(Boc)-OH, Fmoc-Val-OH, Fmoc-Lys(Fmoc)-OH, Fmoc-Asp(tBu)-OH, Fmoc-Asp-OtBu, Fmoc-Glu(tBu)-OH, Fmoc-Glu-OtBu, Fmoc-Gly-OH, Fmoc-Tyr(tBu)-OH, Fmoc-Ile-OH, Fmoc-Ser-OH, Fmoc-Nle-OH, Fmoc-His(Trt)-OH, Fmoc-D-Phe-OH, Fmoc-Phe-OH, Fmoc-Arg(Pbf)-OH, Fmoc-Trp(Boc)-OH, Fmoc-Pro-OH, Fmoc-Cys(Trt)-OH and Fmoc-Asn(Trt)-OH. Rink Amide AM LL resin was purchased from Novabiochem. Wang resin was purchased from Iris BIOTECH GMBH. Analytical RP-HPLC was performed with an Ultimate 3000 Rapid Separation LC-MS System (DAD-3000RS diode array detector) using an Acclaim RSLC 120 C18 column (2.2 μm, 120 Å, 3×50 mm, flow 1.2 mL/min) from Dionex. Data recording and processing was done with Dionex Chromeleon Management System Version 6.80 (analytical RP-HPLC). All RP-HPLC were using HPLC-grade acetonitrile and Milli-Q deionized water. The elution solutions were: A: MilliQ deionized water containing 0.05% TFA; D: MilliQ deionized water/acetonitrile (10:90, v/v) containing 0.05% TFA (except for analyses of Fmoc deprotection in solution, see section 3 below). Preparative RP-HPLC was performed with a Waters automatic Prep LC Controller System containing the four following modules: Waters2489 UV/Vis detector, Waters2545 pump, Waters Fraction Collector Ill and Waters 2707 Autosampler. A Dr. Maisch GmbH Reprospher column (C18-DE, 100×30 mm, particle size 5 μm, pore size 100 Å, flow rate 40 mL/min) was used. Compounds were detected by UV absorption at 214 nm using a Waters 248 Tunable Absorbance Detector. Data recording and processing was performed with Waters ChromScope version 1.40 from Waters Corporation. All RP-HPLC were using HPLC-grade acetonitrile and Milli-Q deionized water. The elution solutions were: A MilliQ deionized water containing 0.1% TFA; D MilliQ deionized water/acetonitrile (10:90, v/v) containing 0.1% TFA. MS spectra, recorded on a Thermo Scientific LTQ OrbitrapXL, were provided by the MS analytical service of the Department of Chemistry, Biochemistry and Pharmaceutical Sciences at the University of Bern (group PD Dr. Stefan Schurch).

### Solid-Phase Peptide Synthesis (SPPS)

### SPPS of G1KL

All peptide dendrimers were synthesized using standard 9-fluorenylmethoxycarbonyl (Fmoc) Solid Phase Peptide Synthesis. All syntheses of peptide dendrimers were performed at 60° C. (or room temperature) under nitrogen bubbling. All peptide dendrimers were synthesized using Rink Amide LL resin (0.26-0.29 mmol/g). Branching points consisted of Fmoc-Lys(Fmoc)-OH to obtained two free amines after Fmoc deprotection (mainchain and sidechain)

Resin was firstly deprotected twice one minute and four minutes using the corresponding deprotection cocktail. For each amino acid, a doubling coupling was performed (twice eight minutes) using for each coupling 3 mL of 0.2 M of the corresponding Fmoc protected amino acid in DMF, 1.5 mL of 0.5 M Oxyma in DMF and 2 mL of 0.5 M DIC in DMF. Deprotection steps (one minute and four minutes) were achieved using the corresponding deprotection solution.

After the SPPS, peptide dendrimers were cleaved from the resin at room temperature using 7 mL of a mixture trifluoroacetic acid/triisopropylsilane/mQ water (TFA/TIS/H₂O) with the corresponding ratios 94/5/1 for three. Peptides were then precipitated using approximatively 25 mL of cold terbutylmethyl ether and centrifuged 10 minutes at 4400 rpm. Supernatant was removed and peptides were dried with argon before lyophilization. All peptides were obtained as TFA salts.

### SPPS of G2KL

Syntheses of G2KL were performed at room temperature with the same reagents as described above with a mechanical stirrer and branching was made using Fmoc-Lys(Fmoc)-OH. Double deprotections were performed during 2×10 minutes. Double coupling was performed during 2×1 hour for the three first amino acids and first generation, and during 3×1 hour for the second generation. Branching points consisted of Fmoc-Lys(Fmoc)-OH to obtained two free amines after Fmoc deprotection (mainchain and sidechain). Same conditions were used for the cleavage as described above.

### SPPS of Linear Peptides

All peptides were synthesized using standard 9-fluorenylmethoxycarbonyl (Fmoc) Solid Phase Peptide Synthesis. All syntheses of linear peptides were performed at 60° C. (or 90° C.) under nitrogen bubbling. All peptides were synthesized using Rink Amide LL resin (0.26-0.29 mmol/g) except for Bivaluridin for which Wang resin (1.2 mmol/g) was used to obtain carboxylic acid function at the C-terminus.

Resin was firstly deprotected twice one minute and four minutes using the corresponding deprotection cocktail. For each amino acid, a doubling coupling was performed (twice eight minutes) using for each coupling 3 mL of 0.2 M of the corresponding Fmoc protected amino acid in DMF, 1.5 mL of 0.5 M Oxyma in DMF and 2 mL of 0.5 M DIC in DMF. Deprotection steps (one minute and four minutes) were achieved using the corresponding deprotection solution.

For syntheses at 90° C., coupling time was 2×4 minutes and deprotection times were 0.5 and 2.5 minutes.

For Bivalirudin, first amino acid coupling was performed with DMAP (0.2 eq. in DMF) as coupling reagents due to the carboxyl C-terminus.

After the SPPS, peptides were cleaved from the resin at room temperature using 7 mL of a mixture trifluoroacetic acid/triisopropylsilane/mQ water (TFA/TIS/H₂O) with the corresponding ratios 94/5/1 for three hours or using 7 mL of the mixture TFA/TIS/DODT/H₂O with the corresponding ratios 94/2.5/2.5/1 for Hexapeptide 5. Peptides were then precipitated using approximatively 25 mL of cold terbutylmethyl ether and centrifuged 10 minutes at 4400 rpm. Supernatant was removed and peptides were dried with argon before lyophilization and/or purification. All peptides were obtained as TFA salts.

### Fmoc Deprotection in Solution

50 mg of Fmoc-Lys(Boc)-OH, Fmoc-Phe-OH or Fmoc-PEG-OH were dissolved in the corresponding deprotection condition in a total volume of 500 μL. Deprotection conditions used in DMF were 20% v/v piperidine, 25% v/v dipropylamine, 5% w/v piperazine+2% v/v DBU, 2% v/v DBU, 25% v/v dipropylamine+3% w/v piperazine, 25% v/v diethylamine, 25% v/v diisopropylamine and 25% diisobutylamine. Reaction mixtures were stirred during 30 minutes at room temperature. After the reaction and for each condition, 10 μL were diluted in MeCN for a final volume of 1 mL.

All samples were analyzed by analytical RP-HPLC-MS using solvents B (100 mQ water+0.1% formic acid) and C (90% MeCN+10% mQ water+0.1% formic acid) with a gradient 100% B to 100% C in 7 minutes.

### Analytical Data

G1KL (20% v/v Piperidine) was obtained as crude white solid after lyophilization (90.5 mg, 72.5%). Analytical RP-HPLC: t_{R}=2.11 min (A/D 100:0 to 0:100 in 7.50 min, λ=214 nm). HRMS (ESI+): C₄₂H₈₄N₁₂O₇ calc./obs. 869.66/869.66 Da [M+H]⁺.

G1KL (5% w/v Piperazine+2% v/v DBU) was obtained as crude white solid after lyophilization (30.4 mg, 26.2%). Analytical RP-HPLC: t_{R}=1.85 min (A/D 100:0 to 0:100 in 7.50 min, λ=214 nm). HRMS (ESI+): C₄₂H₈₄N₁₂O₇ calc./obs. 869.66/869.66 Da [M+H]⁺.

G1KL (20% v/v Diisopropylamine) was obtained as crude white solid after lyophilization (0.3 mg, 0.0%, traces). Analytical RP-HPLC: t_{R}=−min (A/D 100:0 to 0:100 in 7.50 min, λ=214 nm). HRMS (ESI+): C₄₂H₈₄N₁₂O₇ calc./obs. 869.66/869.66 Da [M+H]⁺.

G1KL (20% v/v Dipropylamine) was obtained as crude white solid after lyophilization (42.2 mg, 35.2%). Analytical RP-HPLC: t_{R}=1.79 min (A/D 100:0 to 0:100 in 7.50 min, λ=214 nm). HRMS (ESI+): C₄₂H₈₄N₁₂O₇ calc./obs. 869.66/869.66 Da [M+H]⁺.

G1KL (25% v/v Dipropylamine) was obtained as crude white solid after lyophilization (82.4 mg, 64.5%). Analytical RP-HPLC: t_{R}=1.88 min (A/D 100:0 to 0:100 in 7.50 min, λ=214 nm). HRMS (ESI+): C₄₂H₈₄N₁₂O₇ calc./obs. 869.66/869.66 Da [M+H]⁺.

G2KL (20% v/v Piperidine) was obtained as crude white solid after lyophilization (214.4 mg, 64.9%). Analytical RP-HPLC: t_{R}=2.35 min (A/D 100:0 to 0:100 in 7.50 min, λ=214 nm). HRMS (ESI+): C₁₀₂H₂₀₀N₂₈O₁₇ calc./obs. 2090.56/2090.56 Da [M+H]⁺.

G2KL (5% w/v Piperazine+2% DBU) was obtained as crude white solid after lyophilization (189.9 mg, 53.9%). Analytical RP-HPLC: t_{R}=2.32 min (A/D 100:0 to 0:100 in 7.50 min, λ=214 nm). HRMS (ESI+): C₁₀₂H₂₀₀N₂₈O₁₇ calc./obs. 2090.56/2090.56 Da [M+H]⁺.

G2KL (20% v/v Dipropylamine) was obtained as crude white solid after lyophilization (134.4 mg, 42.2%). Analytical RP-HPLC: t_{R}=2.40 min (A/D 100:0 to 0:100 in 7.50 min, λ=214 nm). HRMS (ESI+): C₁₀₂H₂₀₀N₂₈O₁₇ calc./obs. 2090.56/2090.56 Da [M+H]⁺.

G2KL (25% v/v Dipropylamine) was obtained as crude white solid after lyophilization (151.4 mg, 46.4%). Analytical RP-HPLC: t_{R}=2.38 min (A/D 100:0 to 0:100 in 7.50 min, λ=214 nm). HRMS (ESI+): C₁₀₂H₂₀₀N₂₈O₁₇ calc./obs. 2090.56/2090.56 Da [M+H]⁺.

VKDGYI (SEQ ID NO: 3) (20% v/v Piperidine) was obtained as white solid after preparative RP-HPLC (3.2 mg, 4.5%). Analytical RP-HPLC: t_{R}=1.92 min (A/D 100:0 to 0:100 in 7.50 min, λ=214 nm). HRMS (ESI+): C₃₂H₅₂N₈O₉ calc./obs. 693.39/693.39 Da [M+H]⁺.

VKDGYI (SEQ ID NO: 3) (20% v/v Piperidine+0.5 M Oxyma) was obtained as crude white solid after lyophilization (10.8 mg, 16.8%). Analytical RP-HPLC: t_{R}=1.99 min (A/D 100:0 to 0:100 in 7.50 min, λ=214 nm). HRMS (ESI+): C₃₂H₅₂N₈O₉ calc./obs. 693.39/693.39 Da [M+H]⁺.

VKDGYI (SEQ ID NO: 3) (5% w/v Piperazine+2% v/v DBU) was obtained as crude white solid after lyophilization (5.7 mg, 0.0%). Analytical RP-HPLC: t_{R}=−min (A/D 100:0 to 0:100 in 7.50 min, λ=214 nm). HRMS (ESI+): C₃₂H₅₂N₈O₉ calc./obs. 693.39/− Da [M+H]⁺. (No compound observed).

VKDGYI (SEQ ID NO: 3) (2% v/v DBU) was obtained as crude white solid after lyophilization (36.6 mg, 25.7%). Analytical RP-HPLC: t_{R}=2.05 min (A/D 100:0 to 0:100 in 7.50 min, λ=214 nm). HRMS (ESI+): C₃₂H₅₂N₈O₉ calc./obs. 693.39/693.39 Da [M+H]⁺.

VKDGYI (SEQ ID NO: 3) (20% v/v Dipropylamine) was obtained as crude white solid after lyophilization (37.3 mg, 49.3%). Analytical RP-HPLC: t_{R}=1.84 min (A/D 100:0 to 0:100 in 7.50 min, λ=214 nm). HRMS (ESI+): C₃₂H₅₂N₈O₉ calc./obs. 693.39/693.39 Da [M+H]⁺.

VKDGYI (SEQ ID NO: 3) (25% v/v Dipropylamine) was obtained as white solid after preparative RP-HPLC (11.5 mg, 16.0%). Analytical RP-HPLC: t_{R}=1.20 min (A/D 100:0 to 0:100 in 3.5 min, λ=214 nm). MS (ESI+): C₃₂H₅₂N₈O₉ calc./obs. 693.39/693.42 Da [M+H]⁺.

VKDGYI (SEQ ID NO: 3) (25% v/v Dipropylamine) crude after lyophilization (39.6 mg, 52.9%): Analytical RP-HPLC: t_{R}=1.96 min (A/D 100:0 to 0:100 in 7.50 min, λ=214 nm). HRMS (ESI+): C₃₂H₅₂N₈O₉ calc./obs. 693.39/693.39 Da [M+H]⁺.

VKDGYI (SEQ ID NO: 3) (25% v/v Dipropylamine, 90° C.) was obtained as crude white solid after lyophilization (26.8 mg, 33.5%). Analytical RP-HPLC: t_{R}=2.01 min (A/D 100:0 to 0:100 in 7.50 min, λ=214 nm). HRMS (ESI+): C₃₂H₅₂N₈O₉ calc./obs. 693.39/693.39 Da [M+H]⁺.

VKD(β)GYI (SEQ ID NO: 11) was obtained as white solid after preparative RP-HPLC (6.4 mg, 14.2%). Analytical RP-HPLC: t_{R}=1.19 min (A/D 100:0 to 0:100 in 2.2 min, λ=214 nm). MS (ESI+): C₃₂H₅₂N₈O₉ calc./obs. 693.39/693.42 Da [M+H]⁺.

GDGAKF (SEQ ID NO: 4) (20% v/v Piperidine) was obtained as crude white solid after lyophilization (40.6 mg, 40.9%). Analytical RP-HPLC: t_{R}=1.75 min (A/D 100:0 to 0:100 in 7.50 min, λ=214 nm). HRMS (ESI+): C₂₆H₄₀N₈O₈ calc./obs. 593.30/593.30 Da [M+H]⁺.

GDGAKF (SEQ ID NO: 4) (25% v/v Dipropylamine) was obtained as crude white solid after lyophilization (38.9 mg, 49.2%). Analytical RP-HPLC: t_{R}=1.76 min (A/D 100:0 to 0:100 in 7.50 min, λ=214 nm). HRMS (ESI+): C₂₆H₄₀N₈O₈ calc./obs. 593.30/593.30 Da [M+H]⁺.

VKDRYI (SEQ ID NO: 5) (20% v/v Piperidine) was obtained as crude white solid after lyophilization (44.0 mg, 40.3%). Analytical RP-HPLC: t_{R}=1.99 min (A/D 100:0 to 0:100 in 7.50 min, λ=214 nm). HRMS (ESI+): C₃₆H₆₁N₁₁O₉ calc./obs. 792.47/792.47 Da [M+H]⁺.

VKDRYI (SEQ ID NO: 5) (25% v/v Dipropylamine) was obtained as crude white solid after lyophilization (44.2 mg, 43.4%). Analytical RP-HPLC: t_{R}=2.00 min (A/D 100:0 to 0:100 in 7.50 min, λ=214 nm). HRMS (ESI+): C₃₆H₆₁N₁₁O₉ calc./obs. 792.47/792.47 Da [M+H]⁺.

GDRAKF (SEQ ID NO: 6) (20% v/v Piperidine) was obtained as crude white solid after lyophilization (44.2 mg, 50.6%). Analytical RP-HPLC: t_{R}=1.84 min (A/D 100:0 to 0:100 in 7.50 min, λ=214 nm). HRMS (ESI+): C₃₀H₄₈N₁₀O₉ calc./obs. 693.36/693.39 Da [M+H]⁺.

GDRAKF (SEQ ID NO: 6) (25% v/v Dipropylamine) was obtained as crude white solid after lyophilization (52.9 mg, 62.5%). Analytical RP-HPLC: t_{R}=1.86 min (A/D 100:0 to 0:100 in 7.50 min, λ=214 nm). HRMS (ESI+): C₃₀H₄₈N₁₀O₉ calc./obs. 693.36/693.39 Da [M+H]⁺.

VKDCYI (SEQ ID NO: 7) (20% v/v Piperidine) was obtained as crude white solid after lyophilization (46.2 mg, 53.1%). Analytical RP-HPLC: t_{R}=2.23 min (A/D 100:0 to 0:100 in 7.50 min, λ=214 nm). HRMS (ESI+): C₃₃H₅₄N₈O₉S calc./obs. 739.37/739.38 Da [M+H]⁺.

VKDCYI (SEQ ID NO: 7) (25% v/v Dipropylamine) was obtained as crude white solid after lyophilization (42.7 mg, 48.0%). Analytical RP-HPLC: t_{R}=2.23 min (A/D 100:0 to 0:100 in 7.50 min, λ=214 nm). HRMS (ESI+): C₃₃H₅₄N₈O₉S calc./obs. 739.37/739.38 Da [M+H]⁺.

VKDAYI (SEQ ID NO: 8) (20% v/v Piperidine) was obtained as crude white solid after lyophilization (42.7 mg, 54.7%). Analytical RP-HPLC: t_{R}=2.09 min (A/D 100:0 to 0:100 in 7.50 min, λ=214 nm). HRMS (ESI+): C₃₃H₅₄N₈O₉ calc./obs. 707.40/707.41 Da [M+H]⁺.

VKDAYI (SEQ ID NO: 8) (25% v/v Dipropylamine) was obtained as crude white solid after lyophilization (40.4 mg, 51.3%). Analytical RP-HPLC: t_{R}=2.09 min (A/D 100:0 to 0:100 in 7.50 min, λ=214 nm). HRMS (ESI+): C₃₃H₅₄N₈O₉ calc./obs. 707.40/Da [M+H]⁺.

VKEGYI (SEQ ID NO: 9) (20% v/v Piperidine) was obtained as crude white solid after lyophilization (34.8 mg, 47.7%). Analytical RP-HPLC: t_{R}=1.99 min (A/D 100:0 to 0:100 in 7.50 min, λ=214 nm). HRMS (ESI+): C₃₃H₅₄N₈O₉ calc./obs. 707.40/707.41 Da [M+H]⁺.

VKEGYI (SEQ ID NO: 9) (5% w/v Piperazine+2% v/v DBU) was obtained as crude white solid after lyophilization (38.2 mg, 52.4%). Analytical RP-HPLC: t_{R}=1.90 min (A/D 100:0 to 0:100 in 7.50 min, λ=214 nm). HRMS (ESI+): C₃₃H₅₄N₈O₉ calc./obs. 707.40/707.41 Da [M+H]⁺.

VKEGYI (SEQ ID NO: 9) (20% v/v Dipropylamine) was obtained as crude white solid after lyophilization (32.3 mg, 44.3%). Analytical RP-HPLC: t_{R}=1.98 min (A/D 100:0 to 0:100 in 7.50 min, λ=214 nm). HRMS (ESI+): C₃₃H₅₄N₈O₉ calc./obs. 707.40/707.41 Da [M+H]⁺.

VKEGYI (SEQ ID NO: 9) (20% v/v Dipropylamine+0.5 M Oxyma) was obtained as crude white solid after lyophilization (36.5 mg, 50.0%). Analytical RP-HPLC: t_{R}=1.93 min (A/D 100:0 to 0:100 in 7.50 min, λ=214 nm). HRMS (ESI+): C₃₃H₅₄N₈O₉ calc./obs. 707.40/707.41 Da [M+H]⁺.

Bivaluridin (25% v/v Dipropylamine) was obtained as foamy white solid after preparative RP-HPLC (93.3 mg, 38.7%). Analytical RP-HPLC: t_{R}=1.52 min (A/D 100:0 to 0:100 in 3.50 min, λ=214 nm). HRMS (ESI+): C₉₈H₁₃₈N₂₄O₃₃ calc./obs. 2179.99/2179.99 Da [M+H]⁺.

Bivaluridin (20% v/v Piperidine) was obtained as foamy white solid after preparative RP-HPLC (111.6 mg, 46.3%). Analytical RP-HPLC: t_{R}=1.52 min (A/D 100:0 to 0:100 in 3.50 min, λ=214 nm). HRMS (ESI+): C₉₈H₁₃₈N₂₄O₃₃ calc./obs. 2179.99/2179.99 Da [M+H]⁺.

### LIST OF REFERENCES

R1: Merrifield, R. B., J. Am. Chem. Soc., 1963, 85(14), 2149-2154.
R2: W. Li, N. M. O'Brien-Simpson, M. A. Hossain, J. D. Wade, W. Li, N. M. O'Brien-Simpson, M. A. Hossain and J. D. Wade, Aust. J. Chem., 2019, 73, 271-276.
R3: Ralhan, K., KrishnaKumar, V. G., & Gupta, S., RSC Advances, 2015, 5(126), 104417-104425.
R4: O. F. Luna, J. Gomez, C. Cerdenas, F. Albericio, S. H. Marshall and F. Guzmãn, Molecules, 2016, 21, 1542.
R5: T. Michels, R. Dölling, U. Haberkorn and W. Mier, Org. Lett., 2012, 14, 5218-5221.
R6: C.-C. Chen, B. Rajagopal, X. Y. Liu, K. L. Chen, Y.-C. Tyan, F. Lin and P.-C. Lin, Amino Acids, 2014, 46, 367-374.
R7: V. Rodŕiguez, H. Pineda, N. Ardila, D. Insuasty, K. Cárdenas, J. Román, M. Urrea, D. Ramírez, R. Fierro, Z. Rivera and J. García, Int. J. Pept. Res. Ther., 2020, 26, 585-587.
R8: G. Martelli, P. Cantelmi, C. Palladino, A. Mattellone, D. Corbisiero, T. Fantoni, A. Tolomelli, M. Macis, A. Ricci, W. Cabri and L. Ferrazzano, Green Chem., 2021, 23, 8096-8107.
R9: Palomo, J. M. Rsc Advances, 2014, 4(62), 32658-32672.
R10: Isidro-Llobet, A., Alvarez, M., & Albericio, F., Chem. Rev., 2009, 109(6), 2455-2504.
R11: Insuasty Cepeda, D. S., Pineda Castañeda, H. M, Rodríguez Mayor, A. V., García Castañeda, J. E., Maldonado Villamil, M., Fierro Medina, R., & Rivera Monroy, Z. J., Molecules, 2019, 24(7), 1215.
R12: Mant, Colin T. et al. “HPLC analysis and purification of peptides. “Peptide Characterization and Application Protocols”. Humana Press, 2007, p. 3-55.

1. A method for the preparation of peptides via solid-phase peptide synthesis, wherein
a. an Fmoc protected amino acid building block linked to a resin R-AA-(AA)ₙ-PF, with
R being a resin
AA being an amino acid building block
PF being an Fmoc protecting group
n being the number of coupling cycles,
b. is deprotected in a deprotection step prior to a coupling step, using dibutylamine or dipropylamine, yielding R-AA-(AA)ₙ, wherein
c. in said coupling step, another amino acid building block AA-P is coupled to R-AA-(AA)ₙ, yielding R-AA-(AA)ₙ₊₁-P until a final coupling cycle n_{end}, wherein P is a protecting group of the amino acid building block AA at the N-Terminus or PF.

2. The method according to claim 1, wherein at least one AA comprises aspartic acid.

3. The method according to claim 1, wherein said deprotection step is performed with dipropylamine.

4. The method according to claim 1, wherein the amount of dipropylamine is 10 to 50% (v/v), particularly 20%-to 40 (v/v), more particularly 25% to 35% (v/v).

5. The method according to claim 1, wherein the method is conducted at 10 to 90° C., particularly 65 to 90° C., more particularly 75 to 90° C.

6. The method according to claim 1, wherein the AA-P is activated in an activation step prior to said coupling step.

7. The method according to claim 1, wherein the deprotection step and the coupling step can be repeated until a desired peptide length is reached, which is not more than 60 amino acids.

8. The method according to claim 1, wherein n_{end} is the final coupling cycle at which the desired peptide length is reached.

9. The method according to claim 1, wherein the method comprises a final deprotection step after n_{end} coupling cycles, wherein R-AA-(AA)_{nend}-P is deprotected in said deprotection step yielding R-AA-(AA)_{nend}.

10. The method according to claim 1, wherein the method comprises a cleavage step after n_{end} coupling cycles, wherein R-AA-(AA)_{nend} is cleaved from the resin R, yielding AA-(AA)_{nend}.

11. The method according to claim 11, comprising a purification step, wherein AA-(AA)_{nend} is purified.

12. The method according to claim 1, wherein P is PF.


SEQUENCE LISTING (11 sequences)

SEQ ID NO: 1 - protein, 7 aa, synthetic construct
  SITE 5: note = Branching L-Lysine
  SITE 7: note = C-terminus is carboxamide
KLKLKKL

SEQ ID NO: 2 - protein, 17 aa, synthetic construct
  SITE 9: note = Branching L-Lysine
  SITE 12: note = Branching L-Lysine
  SITE 15: note = Branching L-Lysine
  SITE 17: note = C-terminus is carboxamide
KLKLKLKLKKLKKLKKL

SEQ ID NO: 3 - protein, 6 aa, synthetic construct
  SITE 6: note = C-terminus is carboxamide
VKDGYI

SEQ ID NO: 4 - protein, 6 aa, synthetic construct
  SITE 6: note = C-terminus is carboxamide
GDGAKF

SEQ ID NO: 5 - protein, 6 aa, synthetic construct
  SITE 6: note = C-terminus is carboxamide
VKDRYI

SEQ ID NO: 6 - protein, 6 aa, synthetic construct
  SITE 6: note = C-terminus is carboxamide
GDRAKF

SEQ ID NO: 7 - protein, 6 aa, synthetic construct
  SITE 6: note = C-terminus is carboxamide
VKDCYI

SEQ ID NO: 8 - protein, 6 aa, synthetic construct
  SITE 6: note = C-terminus is carboxamide
VKDAYI

SEQ ID NO: 9 - protein, 6 aa, synthetic construct
  SITE 6: note = C-terminus is carboxamide
VKEGYI

SEQ ID NO: 10 - protein, 20 aa, synthetic construct
  SITE 1: note = D-Phenylalanine
FPRPGGGGNGDFEEIPEEYL

SEQ ID NO: 11 - protein, 6 aa, synthetic construct
  VARIANT 4: note = beta-glycine
VKDGYI